(19) United States
(12) Patent Application Publication
(10) Pub. No.: US 2018/0214383 A1
(43) Pub. Date: Aug. 2, 2018

(54) PRINTING DRUG TABLETS WITH FULLY CUSTOMIZABLE RELEASE PROFILES FOR PERSONALIZED MEDICINE

(71) Applicant: National University of Singapore, Singapore (SG)

(72) Inventors: Yajuan Sun, Singapore (SG); Siow Ling Soh, Singapore (SG)

(21) Appl. No.: 15/742,754

(22) PCT Filed: Jul. 11, 2016
(86) PCT No.: PCT/SG2016/050321

§ 371 Date: Jan. 8, 2018

Related U.S. Application Data
(60) Provisional application No. 62/193,339, filed on Jul. 16, 2015.

(51) Int. Cl. A61K 9/20

(52) U.S. Cl. CPC A61K 9/2072; A61K 9/2095; A61K 9/204

(57) ABSTRACT

Provided herein is a method for producing a dosage form that can be customized according to a patient's needs. In particular, the invention relates to dosage forms comprising an erodible polymer and an active pharmaceutical agent, wherein the erodible polymer is designed to have a specified geometric shape. As described herein, the active agent is released from the dosage form as a function of the geometric shape of the erodible polymer.

### BACKGROUND OF THE INVENTION

Personalized medicine is a medical model that proposes the customization of healthcare, with medical decisions, practices, and/or products being tailored to the individual patient. For example, pharmacy compounding relates to the customized production of a drug product whose various properties (e.g., dose level, ingredient selection, route of administration, etc.) are selected and crafted for an individual patient. Currently, methods of producing drug tablets with release profiles that are truly customizable are limited, challenging, and expensive. Accordingly, there is a significant unmet need for a method of producing fully customizable drug tablets.

### SUMMARY OF THE INVENTION

The present invention provides compositions and methods for customizing the release profile of an active pharmaceutical agent using, among others, three-dimensionally printed templates to form erodible solid polymers containing the active agent, wherein the erodible solid polymers are formed according to the shape of the template. The release profile of the active agent from the erodible solid polymer is determined by, e.g., the geometric shape of the erodible solid polymer. Described herein are dosage forms comprising such erodible solid polymers having specified geometric shapes, as well as methods of using such compositions.

Accordingly, in one aspect, the present invention provides a dosage form, comprising: a) a first erodible polymer having a first three-dimensional geometric shape and comprising at least one active pharmaceutical agent; b) a second erodible polymer that surrounds the first erodible polymer to form an erodible composite, said composite having a second three-dimensional geometric shape having a first end and a second end along a y-axis; and c) a non-erodible housing that encapsulates the erodible composite except at the first end along the y-axis.

In another aspect, the present invention provides a method of delivering a variable dosage form as described herein, wherein, upon contact with a surrounding solvent, the active pharmaceutical agent is released from the first end of the erodible composite as a function of the first geometric shape of the first erodible polymer to deliver a variable dosage to the subject. In further embodiments, the active pharmaceutical agent is released from the first end of the erodible composite as a function of the degree of crosslinking of the polymer.

In a further aspect, the present invention provides a method of producing a variable dosage form, comprising: a) providing a mold having a first three-dimensional geometric shape (e.g., three-dimensionally printing a template having a first three-dimensional geometric shape); b) filling the mold having a first three-dimensional geometric shape with a first solution comprising a first polymer and at least one active pharmaceutical agent; c) polymerizing the first solution comprising the first polymer and the at least one active pharmaceutical agent to form a first solid erodible polymer; d) placing the first solid erodible polymer into a three-dimensionally printed non-erodible housing having an opening on one end; e) filling the housing with a second solution comprising a second polymer; and f) polymerizing the second solution comprising the second polymer, forming an erodible composite that includes the first solid erodible polymer, to form a variable dosage form.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 illustrates a tablet that has been designed to release drug with a custom release profile.

FIGS. 2A and 2B show detailed schematics for two examples of methods for fabricating a drug tablet having a customized release profile. The order represented in FIGS. 2A or 2B need not be performed as shown.

FIG. 3 illustrates an experimental demonstration that the tablet (e.g., the dosage form) is capable of releasing drugs with customizable release profiles. The top row shows examples of geometric shapes (lighter shades in each rectangular housing) formed using 3D printed molds. The middle row shows the expected release profiles from each three-dimensional geometric shape (expected profiles derived from the width, w, of the drug-containing polymer in the mold). The bottom row shows the experimental results using dyes. The solid lines (indicated by solid arrows) show the experimental results for a representative run. The dotted lines (indicated by dashed arrows) represent the expected profile as reproduced from the middle row.

FIG. 4 illustrates that the composition of polymer can be altered to customize the rate of release.

FIG. 5 illustrates release of two drugs at the same time, each with their own unique release profile.

FIG. 6 depicts an example of a dosage form, showing the relative position of an erodible polymer having a triangular geometric shape with respect to the overall dosage form. The x- and y-axis coordinates are shown for relative orientation.

### DETAILED DESCRIPTION OF THE INVENTION

A description of example embodiments of the invention follows.

The present invention provides compositions and methods for customizing the release profile of an active pharmaceutical agent. Generally, a commercially-available three-dimensional (3D) printer is used to print a template having a desired three-dimensional geometric shape, which determines the type of release profile. Because the 3D printer has the flexibility to print any desired three-dimensional shape, the present methods can be used to make fully customizable release profiles.

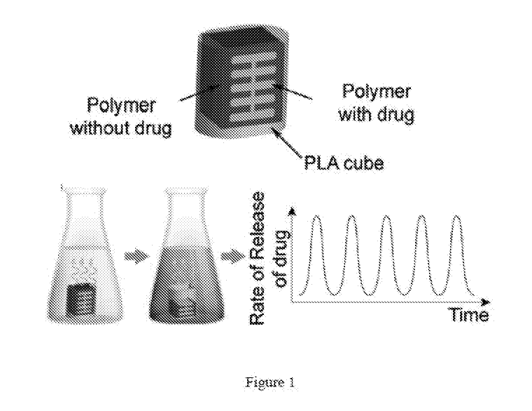

FIG. 1 illustrates the general concept of the present invention. Generally, the dosage forms comprise three components: (1) a surface-eroding polymer (a solid erodible polymer) that contains an active pharmaceutical agent, (2) a surface-eroding polymer (a solid erodible polymer) that does not contain an active pharmaceutical agent, and (3) an impermeable and biodegradable barrier (housing) that protects the sides of the erodible polymer, leaving only one side of the tablet open to the medium (top of FIG. 1). The method includes making customizable shapes of the surface-eroding polymer that contain the drug (component 1 described herein). For example, as shown in FIG. 1, the drug-containing polymer consists of five long bands separated by smaller segments. When the dosage form shown in FIG. 1 (which contains the five long bands separated by smaller segments) is immersed in an aqueous solution, the medium will erode the polymer from the side that is exposed to the solution (as depicted in the flasks shown in FIG. 1). As the polymer erodes gradually, drug releases from the tablet. In this example, five pulses of drug release are obtained with respect to time due to the shape (the five long bands) of the drug-containing polymer.

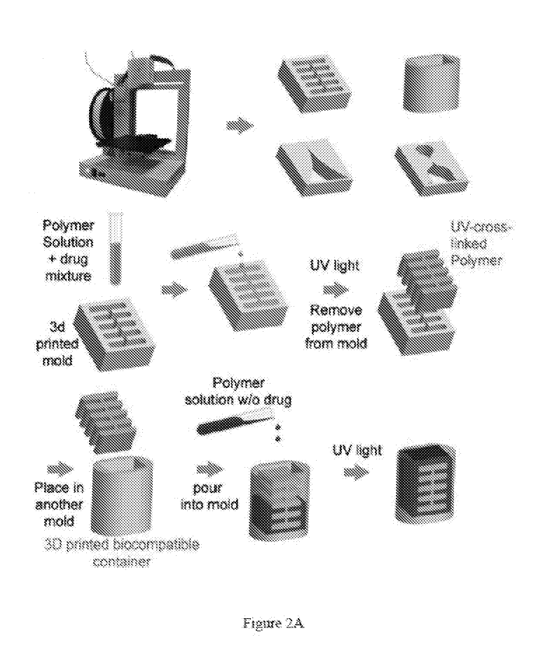

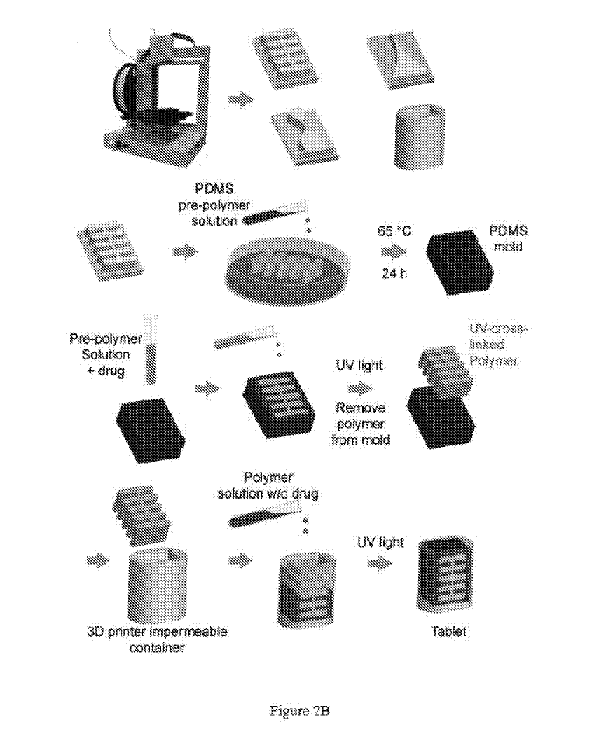

FIGS. 2A and 2B show two examples of a general schematic for fabricating a dosage form (e.g., drug tablet) having a customized release profile. A 3D printer (e.g., one that is commercially available) is used to print a 3D template having a desired geometric shape. The template can be used to form an erodible polymer with a desired three-dimensional shape. For example, as shown in FIG. 2A, a solution, e.g., comprising a polymer and an active pharmaceutical agent is poured into the 3D-printed template and polymerized (e.g., using UV light) to form a solid erodible polymer comprising an active pharmaceutical agent; the polymerized solid erodible drug-containing polymer is removed from the template and placed into a housing. In another example, as shown in FIG. 2B, an embossed template is 3D-printed (using, e.g., acrylonitrile butadiene styrene), which is used to form a mold (made of e.g., PDMS) into which a solution, e.g., comprising a polymer and an active pharmaceutical agent is poured into the mold and polymerized (e.g., using UV light) to form a solid erodible polymer comprising an active pharmaceutical agent. The polymerized solid erodible drug-containing polymer is removed from the mold (formed with the 3D-printed embossed template) and placed into a housing. The housing, which is also printed on a 3D printer, protects the sides of the erodible polymer with impenetrable (but biodegradable) bathers, such that the drug is released only from a specific opening (e.g., the release is one-dimensional). The housing can be made of an impenetrable but biodegradable material such as, e.g., polylactic acid (PLA). The erodible polymer comprising an active pharmaceutical agent is placed inside the housing; the rest of the space (i.e., the void left because the mold may not be of the same shape and size as the housing) is filled with the same erodible polymer, but without any drug. Upon polymerization of the non-drug containing erodible polymer, a composite is formed (the non-drug containing erodible polymer and polymer containing a drug), and the dosage form assembly is complete. The overall shape of the dosage form is determined by the housing. This system allows drugs to be released with any arbitrary release profile with time depending on the shape of the printed mold (the mold that is not the housing), as exemplified in vitro herein. Devices and methods of 3D printing are available and known in the art.

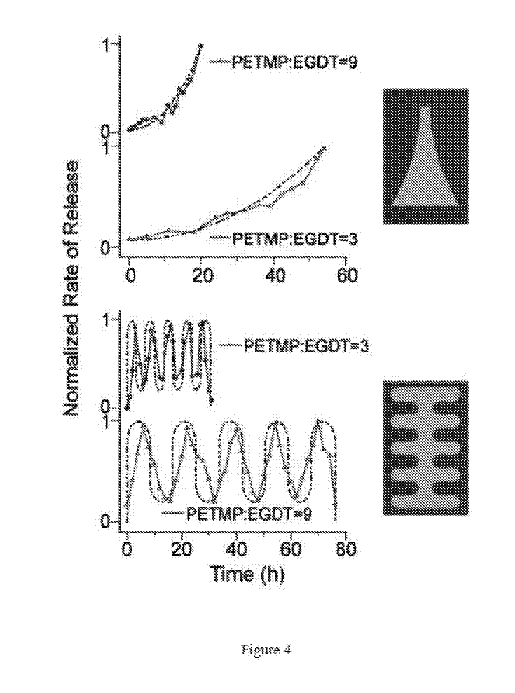

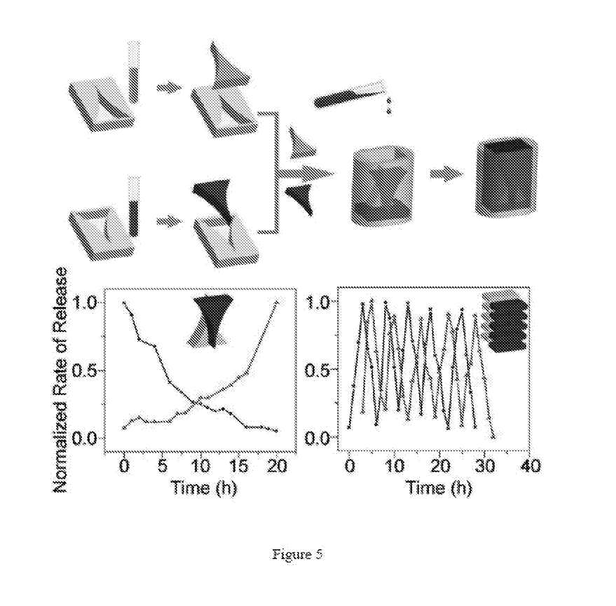

The invention also provides a method to tune the composition of the erodible polymer to either “stretch” (extend) or “compress” (shorten) the period of time that the drug is released (with its desired, unique release profile), as exemplified herein (FIG. 4). In addition, described herein is a method of releasing more than one drug—each with its own unique release profile—from the same dosage form (e.g., tablet), as exemplified herein (FIG. 5).

The present invention is applicable for either mass production of drug tablets, or at a smaller scale that is personalized for the individual patient. For example, in the latter scenario, the present invention can be practiced in a clinical setting where a healthcare provider can decide the desired release profile for the particular patient. The tablet (with this desired profile) can then be fabricated in the same place, and be dispensed to the patient immediately. Thus, in certain aspects, the present invention can be used in the area of personalized medicine, enabling the design of a dosage form to suit the needs of an individual patient.

Accordingly, in one aspect, the present invention provides a dosage form, comprising a first erodible polymer having a first three-dimensional geometric shape and comprising at least one active pharmaceutical agent. The dosage form also comprises a second erodible polymer that surrounds the first erodible polymer to form an erodible composite, said composite having a second three-dimensional geometric shape having a first end and a second end along a y-axis. In a particular embodiment, the second erodible polymer does not contain an active pharmaceutical agent. A non-erodible housing encapsulates the erodible composite except at the first end along the y-axis.

The erodible composite of the resulting dosage form is exposed to the environment (e.g., physiological environment) at the first end (that is, at one end of the dosage form—see, e.g., FIG. 1 showing green wavy arrows from the exposed end of the depicted dosage form). The erodible polymers erode only at the exposed surface, but does not allow drugs from its inner bulk volume to diffuse outward (i.e., the speed of erosion at its surface is faster than the diffusion of drugs within the matrix). Surface-eroding polymers are known and available in the art. Some examples of surface-eroding polymers include polyanhydride and poly(ortho)ester. In one embodiment, the first and second polymers are the same and erode at the same rate. In other embodiments, the first and second polymers are different and erode at different rates. Additionally, using a variety of polymers having different degrees of cross-linking, the composition of the erodible polymer can be varied to either extend (with higher cross-linking) or shorten (with lower cross-linking) the period of time that the drug is released, as exemplified herein (FIG. 4). Thus, while the first geometric shape of the dosage form can be used to determine the release profile, the crosslinking of the polymer can be varied to control how rapidly (or how slowly) the drug is released. Those of skill in the art can determine a suitable surface-erodible polymer having the desired degree of crosslinking.

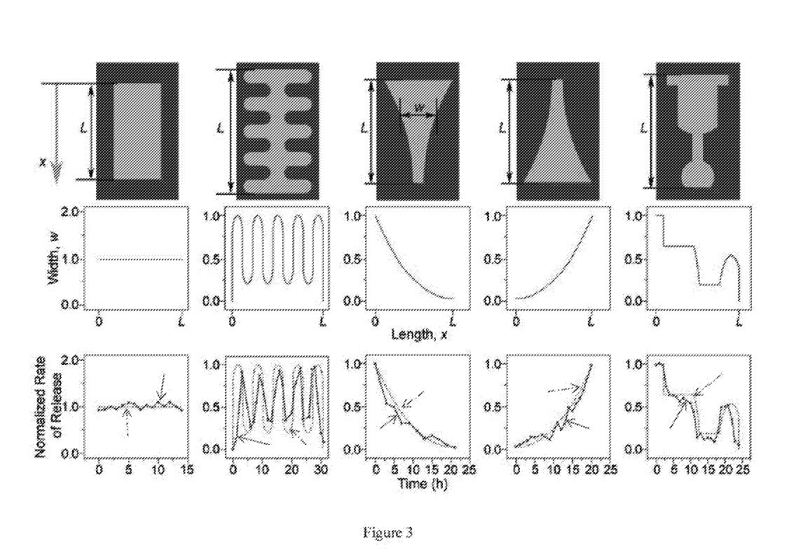

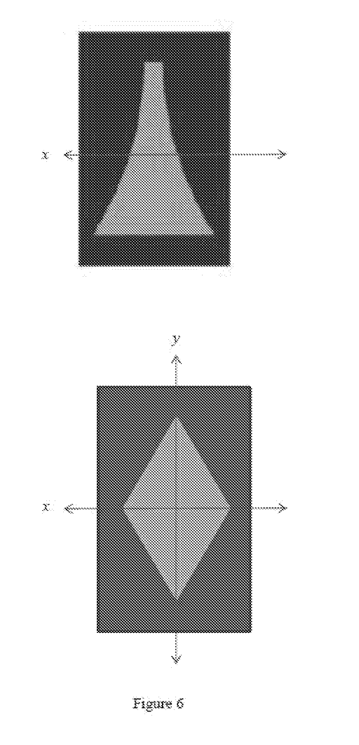

Generally, the first erodible solid polymer having a first three-dimensional geometric shape is positioned in the center of the composite (see, e.g., FIG. 3, top row, showing the position of each first 3D geometric shape—in lighter shading—surrounded by the second erodible polymer—in darker shading) for relatively level erosion of the first erodible polymer. For example, as shown in FIG. 3, the amount of drug released from the first erodible solid polymer is determined by the width of the first erodible solid polymer (as well as its depth, not shown), which correlates to its surface area exposed and in contact with the environment. Thus, it is desirable for the first erodible solid polymer to be positioned “level” within the second erodible solid polymer to produce the desired release profile (see, for example, FIG. 6).

As described herein, the housing of the dosage form is also 3D printed using, e.g., a commercially available 3D printer. The housing serves as a barrier to protect all but one side of the dosage form from eroding such that the drug is release only from a specific opening (the release is one-dimensional). See, e.g., FIG. 2, bottom left “3D printed biocompatible container” having an opening at the top of the depiction. In certain embodiments, the housing is made of a polymer that is non-erodible, but biodegradable (e.g., polylactic acid—PLA; poly(lactic-co-glycolic acid); and dextran-hydroxyethylmethacrylate). Generally, the shape of the overall dosage form takes on the shape of the housing. The housing can be formed into various three-dimensional shapes including, for example, a three-dimensional rectangle, square, or oblong cylinder (such as that shown in FIG. 2, bottom left “container”). In certain embodiments, the outer edges of the housing can be rounded for ease of administration (e.g., easier to swallow).

The first erodible solid polymer having a first geometric shape sits inside the housing, positioned according to an x- and y-axis as depicted in FIG. 6. The erodible solid polymer comprising an active pharmaceutical agent can have a geometric shape and be positioned within the housing such that it is symmetric along a y-axis, or symmetric along an x-axis, or both. Examples of such geometric shapes include is a square box; a rectangular box (as shown, for example in FIG. 3, first illustration from the left in the top row); a cylinder of any aspect ratio with flat ends; a cylinder of any aspect ratio with rounded ends; spherical; ellipsoidal; or a three-dimensional diamond. For example, “spherical” can include a geometric shape that is rounded like an even ball, or a circular disc having a particular thickness, wherein the edge of the disc lies in the y-plane of FIG. 6. In another example, a 3D diamond can include a “flat” diamond shape having a thickness (that is, a disc in the shape of a diamond) positioned in the housing so that it is symmetric along the x- and y-axes (FIG. 6, bottom panel). In other embodiments, the first geometric configuration is asymmetric along a y-axis, or asymmetric along an x-axis, or both. For example, a three-dimensional triangle can be positioned within the housing such that it is symmetric along the y-axis, but asymmetric along the x-axis (see, for example, the triangular shape in FIG. 6 showing symmetry about the y-axis, but asymmetry about the x-axis). The 3D triangular shape includes a “flat” triangle shape having a thickness (that is, a disc in the shape of a triangle) formed from a triangular template like that shown in FIGS. 2A and 2B (one of 4 templates shown in FIGS. 2A and 2B, top right).

In some embodiments, the dosage form can be designed to release more than one active pharmaceutical agent. For example, the first erodible solid polymer having a first three-dimensional geometric shape can be formed from alternating layers of 2 or more drugs such that different drugs can be released sequentially. In a simple scenario, the first erodible polymer can be a three-dimensional rectangle capable of releasing two drugs, each drug layered in an alternating fashion such that erosion of the polymer along the y-axis will release each drug sequentially. In other embodiments, the dosage form further comprises a third solid erodible polymer having a third three-dimensional geometric shape and comprises one or more additional active pharmaceutical agent. The third erodible polymer can be the same polymer as that of the first erodible polymer, or a different polymer that erodes at a similar rate as the first polymer. In various embodiments, the third erodible polymer can be the same shape as the first erodible polymer to produce the same, simultaneous release profile. In one embodiment, the third erodible polymer can be the same shape as the first erodible polymer, but positioned in the housing so as to produce the reverse release profile (see FIG. 5, one 3D triangle positioned upright, and the other inverted). In further embodiments, the third erodible polymer can have a three-dimensional shape that is different from the first geometric shape of the first erodible polymer to produce a different release profile altogether.

In other aspects, the present invention also provides a method of delivering a variable dosage form, comprising administering the variable dosage form as described herein to a subject in need of a treatment, wherein, upon contact with a surrounding solvent, the active pharmaceutical agent is released from the first end of the solid erodible composite as a function of the first geometric shape of the first erodible polymer to deliver a variable dosage to the subject.

In further aspects, the present invention provides a method of producing a variable dosage form as described herein, comprising providing a mold having a first 3D geometric shape, such as by three-dimensionally printing a template have a three-dimensional geometric shape; filling the mold with a first solution comprising a first polymer and at least one active pharmaceutical agent; polymerizing the first solution comprising the first polymer and the at least one active pharmaceutical agent to form a first solid erodible polymer; placing the first solid erodible polymer into a three-dimensionally printed non-erodible housing having an opening on one end; filling the housing with a second solution comprising a second polymer; and polymerizing the second solution comprising the second polymer to form an erodible composite that includes the first solid erodible polymer to form a variable dosage form. In certain embodiments, the method further comprises three-dimensionally printing a template (e.g., an embossed template) having a desired three-dimensional geometric shape. As described herein, a mold is formed using the three-dimensionally printed embossed template having a desired 3D geometric shape. For example, the embossed template is placed in a container with the embossed side up, and a solution containing, e.g., polydimethylsiloxane (PDMS) is poured into the container. Upon curing the solution, a mold having a cavity with the 3D geometric shape of the embossed template is formed. This mold can be used to fill with a solution comprising a polymer and at least one active pharmaceutical agent. See, e.g., FIG. 2B.

In another related aspect, the present invention also provides a method of producing a variable dosage form as described herein, comprising providing a mold having a first three-dimensional geometric shape (e.g., three-dimensionally printing a mold having a first three-dimensional geometric shape); filling the mold having a first 3D geometric shape with a first solution comprising a first erodible polymer and at least one active pharmaceutical agent; polymerizing the first solution comprising the first erodible polymer and the at least one active pharmaceutical agent to form a first solid erodible polymer; placing the first solid erodible polymer into a three-dimensionally printed non-erodible housing having an opening on one end; filling the housing with a second solution comprising a second erodible polymer; and polymerizing the second solution comprising the second erodible polymer forming an erodible composite that includes the first solid erodible polymer to form a variable dosage form. In one embodiment, the mold is 3D-printed. In additional embodiments, the mold comprises a cavity having a three-dimensional geometric shape. As described herein, the 3D-printed mold comprising a cavity having a three-dimensional geometric shape can be used to fill with a solution comprising a polymer and at least one active pharmaceutical agent. See, e.g., FIG. 2A.

As will be appreciated by those of skill in the art, the geometric shape of the erodible polymer comprising an active pharmaceutical agent is determined according to the condition of a patient in need of treatment (i.e., according to a diagnosis made for a patient in need of treatment with a variable dosage form). For example, constant and continuous release can be achieved by, for example, a rectangular shape depicted in the first shape from the left of FIG. 3, top row. In another example, pulses of release can be achieved by a geometric shape depicted in the second shape from the left of FIG. 3, top row. Moreover, the nature of the polymer that forms the drug-containing solid erodible polymer is also determined according to the condition of a patient. For example, if it is desired to achieve a slow release, a polymer having the appropriate degree of crosslinking can be used to achieve the desired rate of release.

### EXAMPLES

Release Rate as a Function of the Geometric Shape of the Erodible Polymer

Materials and Methods

Five three-dimensional geometric shapes were selected for demonstration of release profiles (FIG. 3). First, a 3D printer was used to make an embossed template having the desired three-dimensional geometric shape. Acrylonitrile butadiene styrene (ABS) was used to make the template. Then, a polydimethylsiloxane (PDMS) solution was poured over the embossed template as shown in FIG. 2B. The solution was cured by heating it to 65° C. for 24 h, upon which a PDMC mold having a cavity shaped by the embossed template was formed. The PDMS mold was extracted from the template. For visualization of the release profiles, dyes were using instead of a drug for ease of monitoring the progress of the release using a UV-visible spectrophotometer. Thus, a dye was mixed with a polymer solution, poured into the PDMS mold, and allowed to polymerize (crosslink) under UV light, forming a solid erodible polymer containing the dye. The housing was 3D printed, and the polymerized erodible polymer was placed in the housing; a polymer solution was poured into the housing to fill the void space. Upon polymerization, a “dosage” form containing dye was formed, with the top of the composite exposed. Samples of the solutions were taken at regular time intervals, and were analyzed using a UV-visible spectrophotometer.

The dye-containing polymer was generated as follows: 4-pentenoic anhydride (PNA), pentaerythritol tetrakis(3-mercaptopropionate) (PETMP) and 2,2-(Ethylenedioxy) diethanethiol (EGDT) were mixed together, and 0.1 wt % 1-hydroxycyclohexyl phenyl ketone was then added as the photoinitiator. The mole ratio for PNA and the total amount of both cross-linkers used was 1:1. Two different mole ratios were used in this study—PNA:PETMP: EGDT=1:0.75:0.25, and 1:0.9:0.1. The solution was then purged with nitrogen for three minutes. 6 to 8 mg of a dye (Orange G or Brilliant Blue G) were then added to 0.2 mL of the purged solution, and mixed thoroughly using a sonicator. The dye-loaded solution was then added to fill the cavity in the PDMS mold, and exposed to UV light (365 nm) for 10 minutes. After UV, the polymer was cross-linked. The shape of this polymer was the same as the embossed features of the template printed by the 3D printer. This dye-loaded polymer with the desired 3D geometric shape was then extracted from the mold and placed within a housing made of PLA. This housing was also printed using a 3D printer. The same polymer solution (e.g., the mixture of PNA, PETMP, EGDT, and the photoinitiator), but without any dye, was then added into the housing until it was filled (leaving about 0.5 mm space above the dye-loaded polymer). The housing was then placed under vacuum to remove air bubbles before exposure to UV light (365 nm) for 10 minutes. After curing, the process of fabricating the tablet was complete.

Results

Five different types of release profiles known to be clinically important were demonstrated: the constant, pulsed (e.g., five pulses), decreasing, and increasing profiles (FIG. 3, top row). In addition, in order to demonstrate the versatility of the method, an arbitrary profile was also designed—a profile that consists of some periods of constant release together with increasing and decreasing segments. As shown in FIG. 3, the rates of release of the dye were similar to the expected profiles (i.e., the profiles that were expected after drawing the shapes using the 3D printer as illustrated in the schemes on the top two rows in FIG. 3).

Release Rate as a Function of the Erodible Polymer

Materials and Methods

Two types of cross-linker were used to form the surface-eroding polymer: the pentaerythritol tetrakis (3-mercaptopropionate) (PETMP) or the ethylene glycol-based dithiol (EGDT). Because PETMP is a better cross-linker than EGDT, a higher ratio of PETMP was expected to result in a slower rate of erosion. A PETMP:EGDT ratio of 3 and 9 (i.e., 3:1 or 9:1 of PETMP:EGDT) were examined.

Results

The present study demonstrates that it is possible to change the duration of the release using different erodible polymers. Since the rate-limiting step involves the erosion of the polymer, varying the composition of the polymer can change the rate of erosion. FIG. 4 shows that when, e.g., the ratio of PETMP:EGDT is 3:1, the release is complete at ˜20-30 hours. In contrast, when the ratio the PETMP:EGDT ratio is 9:1, the release is complete at ˜50-80 hours. Notably, the release profiles are the same for both cases (one demonstrated for an increasing profile—upper panel in FIG. 4—and the other demonstrated with five pulses—lower panel in FIG. 4).

Release of More than One Agent from a Dosage Form

Materials and Methods

Two molds of the desired profiles were 3D printed, and two dye-containing polymers of their respective shapes were formed as described in the preceding examples. The two erodible polymers, each containing a dye, were placed together face-to-face, and placed in the 3D printed biodegradable housing (as shown in FIG. 5). The void spaces were filled as described above with the pre-polymer that did not contain the drug (or dye) in the impermeable polymer housing. The assembled dosage form was immersed in a medium and the release profile examined

Results

As demonstrated herein, multiple dyes can be released from a single dosage form (tablet), each dye releasable by its own unique release profile. FIG. 5 shows two examples of incorporating two dyes in a dosage form. Upon immersing the tablet in a medium, the tablet could release both dyes at the same time—each with their specific release profiles—as demonstrated in two scenarios. The first consisted of an increasing and a decreasing profile (left lower graph of FIG. 5). The second consisted of releases with five pulses; however, one of them released earlier than the other such that the peak of release of one dye corresponded to the trough of release of the other dye (e.g., the release profile is the same, but out of phase).

In conclusion, as demonstrated the present key present concepts of the tablet we fabricated: In conclusion, as demonstrated herein, the dosage forms of the present invention can be designed to 1) release drugs with any desired release profile, 2) release drugs with a desired duration of release and 3) release more than one drug simultaneously, each with their own unique and desirable release profile.

The relevant teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

It should also be understood that, unless clearly indicated to the contrary, in any methods described herein that include more than one step or act, the order of the steps or acts of the method is not necessarily limited to the order in which the steps or acts of the method are recited.

1. A dosage form, comprising:
a first erodible polymer having a first three-dimensional geometric shape and comprising at least one active pharmaceutical agent;
a second erodible polymer that surrounds the first erodible polymer to form an erodible composite, said composite having a second three-dimensional geometric shape having a first end and a second end along a y-axis; and
a non-erodible housing that encapsulates the erodible composite except at the first end along the y-axis.

2. The dosage form of claim 1, further comprising a third polymer having a third three-dimensional geometric shape and comprising one or more additional active pharmaceutical agent.

3. The dosage form of claim 1, wherein the non-erodible housing is biodegradable.

4. The dosage form of claim 3, wherein the biodegradable housing comprises a polymer.

5. The dosage form of claim 4, wherein the polymer is polylactic acid (PLA).

6. The dosage form of claim 1, wherein the first geometric shape is symmetric along a y-axis, or symmetric along an x-axis, or both.

7. The dosage form of claim 1, wherein the first geometric shape is asymmetric along a y-axis, or asymmetric along an x-axis, or both.

8. The dosage form of claim 6, wherein the geometric shape is a square box; a rectangular box; a cylinder of any aspect ratio with flat ends; a cylinder of any aspect ratio with rounded ends; spherical; ellipsoidal; or a three-dimensional diamond.

9. The dosage form of claim 7, wherein the geometric shape is a three-dimensional triangle.

10. A method of delivering a variable dosage form comprising administering the variable dosage form to a subject, wherein the dosage form comprises:
a first erodible polymer having a first three-dimensional geometric shape and comprising at least one active pharmaceutical agent;
a second erodible polymer that surrounds the first erodible polymer to form an erodible composite, said composite having a second three-dimensional geometric shape having a first end and a second end along a y-axis, and
a non-erodible housing that encapsulates the erodible composite except at the first end along the y-axis;
wherein, upon contact with a surrounding solvent, the active pharmaceutical agent is released from the first end of the erodible composite as a function of the first geometric shape of the first erodible polymer, to deliver a variable dosage to the subject.

11. The method of claim 10, wherein the dosage form further comprises a third polymer having a third three-dimensional geometric shape and comprising one or more additional active pharmaceutical agent.

12. The method of claim 10, wherein the non-erodible housing is biodegradable.

13. The method of claim 12, wherein the biodegradable housing comprises a polymer.

14. The method of claim 10, wherein the first geometric shape is symmetric along a y-axis, or symmetric along an x-axis, or both.

15. The method of claim 10, wherein the first geometric shape is asymmetric along a y-axis, or asymmetric along an x-axis, or both.

16. The method of claim 14, wherein the geometric shape is a square box; a rectangular box;
a cylinder of any aspect ratio with flat ends; a cylinder of any aspect ratio with rounded ends;
spherical; ellipsoidal; or a three-dimensional diamond.

17. The method of claim 15, wherein the geometric shape is a three-dimensional triangle.

18. A method of producing a variable dosage form, comprising:
a) providing a mold having a first three-dimensional geometric shape;
b) filling the mold with a first solution comprising a first polymer and at least one active pharmaceutical agent;
c) polymerizing the first solution comprising the first polymer and the at least one active pharmaceutical agent to form a first solid erodible polymer;
d) placing the first solid erodible polymer into a three-dimensionally printed non-erodible housing having an opening on one end;
e) filling the housing with a second solution comprising a second polymer; and
f) polymerizing the second solution comprising the second polymer, forming an erodible composite that includes the first solid erodible polymer to form a variable dosage form.

19. The method of claim 18, wherein the first geometric shape of the variable dosage form is determined according to a diagnosis made for a patient in need of treatment with a variable dosage form.

20. The method of claim 18, wherein the mold is formed from a three-dimensionally printed template having the first three-dimensional geometric shape.
